(19) United States
(12) Patent Application Publication
(10) Pub. No.: US 2008/0113345 A1
(43) Pub. Date: May 15, 2008

(54) Predicting Response And Outcome Of Metastatic Breast Cancer Anti-Estrogen Therapy

(75) Inventors: Petronella M.J.J. Berns, Dordrecht (NL); Maurice P.H.M. Jansen, Rotterdam (NL); John A. Foekens, Rotterdam (NL); Johannes G.M. Klijn, Rotterdam (NL)

Correspondence Address: MERCHANT & GOULD PC, P.O. BOX 2903, MINNEAPOLIS, MN, 55402-0903, US

(73) Assignee: Erasmus MC, Rotterdam (NL)

(21) Appl. No.: 10/581,611

(22) PCT Filed: Dec. 3, 2004
(86) PCT No.: PCT/IB04/04405

§ 371 Date: Apr. 13, 2007

Related U.S. Application Data
(60) Provisional application No. 60/527,608, filed on Dec. 5, 2003.

(51) Int. Cl. C07H 21/02; C12Q 1/68; G01N 33/574

(52) U.S. Cl. 435/6; 435/7.23; 536/23.5

(57) ABSTRACT

Gene signatures, specific marker genes, and diagnostic assays for predicting progression free survival and objective response to anti-estrogen, e. g., tamoxifen therapy for recurring breast cancer patients are described.

### BACKGROUND

Resistance to anti-estrogens is one of the major challenges in the treatment of breast cancer. For more than 25 years, the golden standard for the endocrine treatment of all stages of estrogen receptor-positive breast cancer has been tamoxifen (Jordan, 2003, Nat. Rev. Drug Discov., 2:205-13; Osborne, 1998, N. Engl. J Med. 339:1609-18). However, in the advanced setting when metastasis is detected approximately half of the patients with estrogen receptor-α (ER-α) -positive breast tumors will not respond to endocrine treatment, whereas response rates in patients with ER-α-negative primary tumors are very low. Therefore additional biomarkers are needed to identify patients who will not respond and to select patients for various tailored treatments.

In the past 20 years a large number of cell biological factors, other than steroid receptors, has been reported that identify those patients who will benefit from endocrine therapy or fail to respond (for review see Klijn et al.:, 2002, Ingle WRMaJN (ed): Endocrine Therapy in Breast Cancer. New York, Marcel Dekker). Few of these, however, appeared valuable and useful in daily clinical practice. In these individual studies only a limited number of factors have been evaluated simultaneously. Breast cancer is known as a heterogeneous and multifactorial disease, with accumulation of (epi)genetic alterations leading to transformation of normal cells into cancer cells. With the advent of high-throughput quantification of gene-expression, simultaneous assessment of thousands of genes is now possible in a single experiment (Brown et al., 1999, Nat. Genet. 21:33-7; Holloway, et al., 2002, Gynecol. Oncol., 87:8-16, 2002). Gene-expression profiling provides a strategy for discovering gene-expression characteristics that may be useful to predict clinical outcome.

### SUMMARY OF THE INVENTION

Using microarray expression profiling, gene signatures, marker genes, and methods were developed for predicting response or resistance to anti-estrogen, for example, tamoxiphen therapy and predicting outcome for recurring breast cancer patients. Using a gene profile described herein, analysis of a patient's primary breast tumor against the gene profile is predictive of patient response to anti-estrogen, for example, tamoxiphen therapy, for example, tamoxifen therapy for the treatment of recurring breast cancer.

Useful gene signatures for predicting outcome (response or resistance) and progression free survival in recurring breast cancer treated with anti-estrogen, for example, tamoxiphen therapy and include the genes of the 81-gene signature and the 44-gene signature shown in FIG. 2. As shown in FIG. 2A, a Cluster I expression pattern of marker genes correlates with progressive disease; a Cluster II expression pattern of marker genes correlates with Objective Respons. In one embodiment, a set of two or more marker genes is predictive. The gene signature may comprise at least one, and preferably at least two of FN-1, CASP-2, THRAP-2, SIAH-2, DEME-6, TNC, and COX-6C. In a specific embodiment, the gene signature comprises at least one of DEME-6 and CASP2, and at least one of SIAH-2 and TNC.

Gene expression levels can be determined using various known methods including nucleic acid hybridization in microarrays, nucleic acid amplification methods such as quantitative polymerase chain reaction (qPCR), and immunoassay of proteins expressed by the genes of the predictive gene profile. Expression levels and expression level ratios of two or more genes of the predictive gene profile can be determined, for example, using real-time quantitative reverse-transcriptase PCR (qRT-PCR).

The gene signatures of the invention are useful in assays to predict response and/or outcome of anti-estrogen, for example, tamoxiphen therapy for recurring breast cancer. In one embodiment, gene expression is analyzed in a primary breast tumor tissue sample and compared to the expressed gene signature determined from retrospective patient data as described in the Examples below. Sample expression data can be analyzed against a classification algorithm determined from a “training” set of data as described in the Examples below.

In another embodiment, a gene expression ratio of two or more genes, or a threshold expression level of one or more predictive genes is analyzed. In a preferred embodiment, expression of at least one upregulated gene and at least one down regulated gene is analyzed. A ratio of the expression of the upregulated gene to that of the down regulated gene is calculated, where the ratio is predictive of response and/or outcome of anti-estrogen, for example, tamoxiphen therapy for treating recurring breast cancer. The predictive ratio or ratios may be stored in a database for comparison to the test data.

The invention includes diagnostic systems and methods such as arrays containing one or more probes to detect expression of one or more genes of the predictive profile. Preferably, the assay system contains at least one of the genes of the 81-gene signature or of the 44-gene signature shown in FIG. 2. In one embodiment, the system contains two or more of these genes. The assay system may comprise at least one, and preferably at least two of FN-1, CASP-2, THRAP-2, SIAH-2, DEME-6, TNC, and COX-6C. In a specific embodiment, the assay system comprises at least one of DEME-6 and CASP2, and at least one of SIAH-2 and TNC.

The gene signatures of the invention are also useful for identifying lead compounds useful in the treatment of estrogen-dependent recurring breast cancer. Primary estrogen-dependent breast tumor tissue can be contacted with the potential therapeutic drug, and the expression of one or more genes of the gene signature analyzed and compared with an untreated control.

These and other features of the invention are described more fully below.

### BRIEF DESCRIPTION OF THE FIGURES

FIG. 1 is a flow chart showing study design and gene selection procedure.

FIG. 2 A and B show a heat map showing clusters of 46 tumors using the 81-gene signature. Cluster 1 shows gene expression correlated with progressive disease; Cluster 2 shows gene expression correlated with objective response. Genes upregulated are shown in red; those downregulated are shown in green. The genes of 81-gene signature are listed, and those of the 44-gene signature are indicated by bars at the right side of the heat map and also listed. NCBI Accession numbers are shown. Bars side of the heat map show genes linked to apoptosis (black), extracellular matrix (purple), and immune system (blue).

FIG. 3 shows a series of progression free survival graphs as a function of gene-signature classification and traditional factors. Progression free survival curves after start of tamoxifen therapy are shown for the validation set of 66 patients grouped according to the traditional factors based score (panels A and B) or the 44-gene signature (panel C).

FIG. 4 is a plot generated with BRB Array Tools showing chromosomal distribution of genes of the entire set analyzed (14557 genes, red bars) and those of a subset of 6 genes of the signature (blue).

### DESCRIPTION OF THE PREFERRED EMBODIMENTS

### Definitions

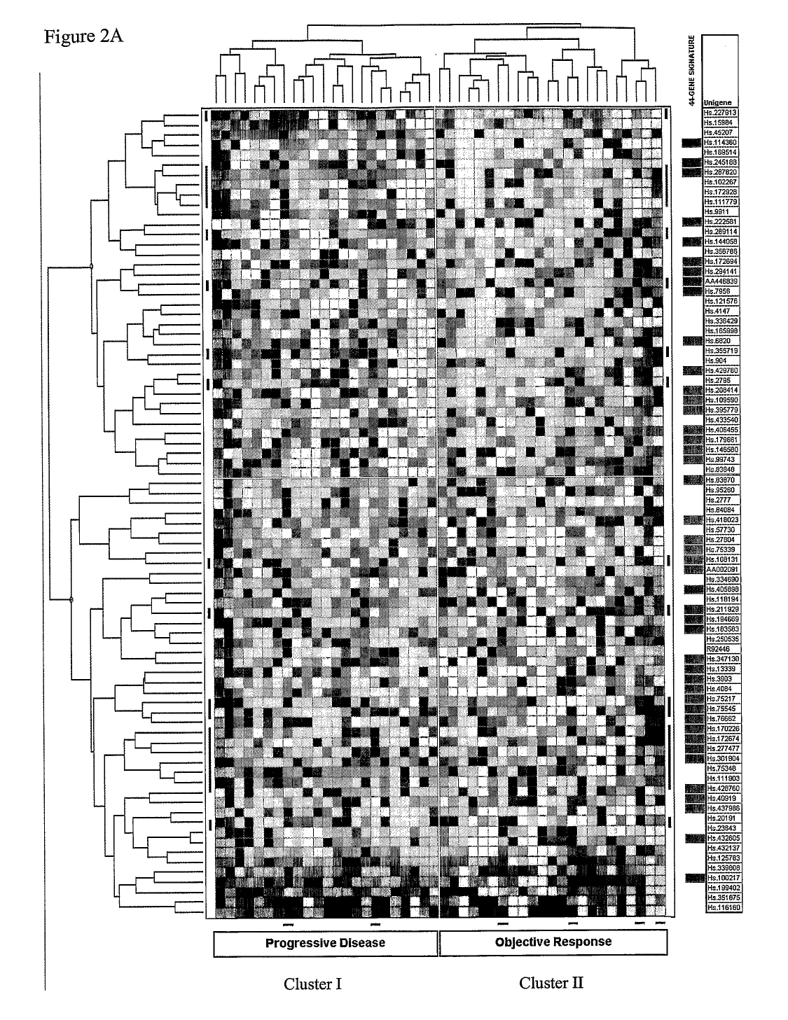

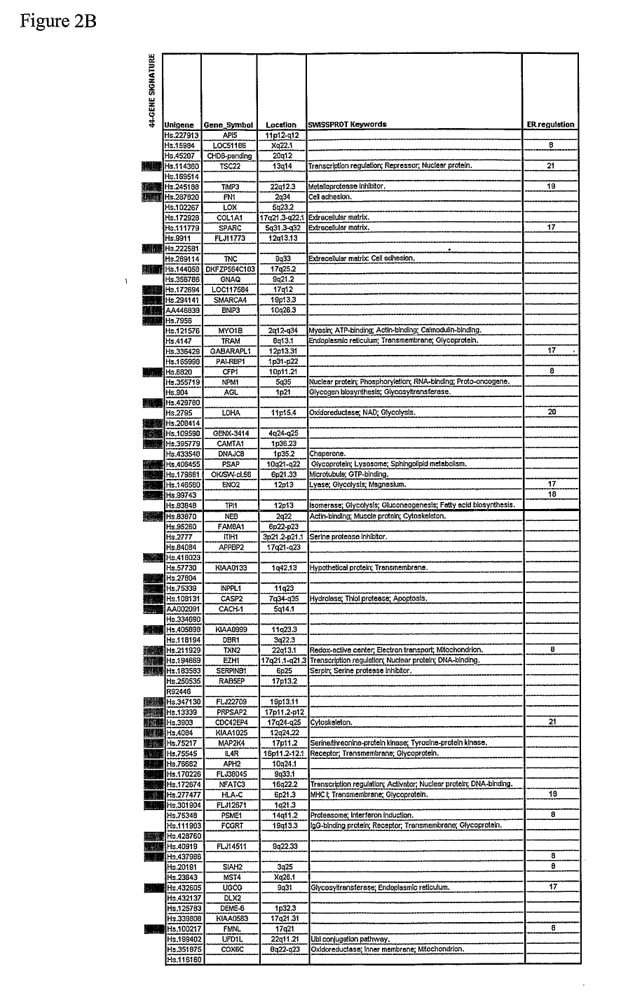

Gene Signature as used herein, refers to a profile of gene expression that correlates with a therapeutic outcome, for example as shown in the heat map of FIG. 2A.

Cluster I and Cluster II gene profiles are shown in FIG. 2A as correlating with progressive disease (Cluster I) and objective response (Cluster II).

Differential expression, as used herein, refers to gene expression in primary breast tumor tissues that differs with a patient's outcome in treatment of recurring breast cancer with anti-estrogen therapy.

Objective response as used herein includes complete remission and partial remission.

Outcome as used herein, refers to Response (complete or partial) or Resistance (progressive disease or stable disease less than 6 months).

Recurring disease or recurring breast cancer is used herein to mean cancer that develops after the primary breast cancer has been removed, for example metastatic breast cancer that occurs after a primar tumor has been excised.

Stable disease refers to patients with no change in disease status, as well as those with no evident tumor reduction of at least 50% or more and those with tumor progression. Patients with stable disease are divided into those with no change (stable disease) for six months or longer, and those with no change (stable disease) for less than six months.

Tumor progression or Progressive Disease, as used herein, is meant to describe growth of about 25% or more tumor mass, or one or more new lesions within a three-month period.

Because patients with stable disease for 6 months or more exhibited a PFS similar to patients with partial remission, these patients were classified as responders to tamoxifen as described in the manual for clinical research and treatment in breast cancer of the European Organization for Research and Treatment of Cancer. European Organization for Research and Treatment of Cancer Breast Cancer Cooperative Group. Manual for clinical research and treatment in breast cancer, Almere, The Netherlands: Excerpta Medica; 2000. p. 116-7.

Clinical benefit was defined in the studies described herein as objective response, including complete and partial remission, and stable disease for six months or more, as described in Ravdin et al., 1992, J. Clin. Oncol. 10:1284-91; Foekens et al., 1994, Br. J Cancer, 70:1217-23; and Robertson et al., 1997, Eur. J Cancer, 33:17749.

Only patients with measurable disease were evaluated in these studies, and selected patients with no change (stable disease), had received tamoxifen at least for a period of 6 months.

### Gene Expression Profiling

Gene expression profiling of retrospective breast cancer tumor tissue using high density cDNA arrays was used herein to generate differential gene expression patterns correlated with patient response and outcome data for treatment of recurrent breast cancer with anti-estrogen, for example, tamoxiphen therapy. Using tumor RNA obtained from a training set of 46 tumors comprising primary tumors from 25 patients exhibiting progressive disease after anti-estrogen, for example, tamoxiphen therapy for recurring breast cancer and primary tumors from 21 patients exhibiting objective response to anti-estrogen, for example, tamoxiphen therapy for recurring breast cancer, differentially expressed genes/ests were identified. Using microarray data analysis tools, (BRB Array Tools), under a significance level of 0.05, a total of 569 and 449 genes were identified as differentially expressed and correlated with progressive disease and objective response, respectively.

81-gene Signature

The overlap of these differentially expressed genes identified an initial signature set of 81 differentially expressed genes having a pattern correlated with progressive disease or objective response for anti-estrogen, for example, tamoxiphen therapy treatment of recurring breast cancer. These 81 genes were classified and subjected to cluster analysis. The results are shown in the heat map of FIG. 2, with a signature pattern of gene expression correlated with predictable response and/or outcome. Genes that were upregulated in the pattern are indicated in red, while genes that were downregulated are shown in green. Gene clustering is also shown by overlapping bars shown on the sides of the expression map.

This 81-gene signature was used to correctly classify retrospective patient samples as having a gene expression pattern correlated with progressive disease or with objective response to anti-estrogen, for example, tamoxiphen therapy in the treatment of metastatic (recurring) breast cancer. 21 of 25 patients with progressive disease and 19 of 21 patients with objective response were correctly classified by this 81-gene signature, as discussed in the Examples below.

44-gene Signature

With further analysis and rank ordering of genes on the basis of significance level, followed by a step-up calculation of correlation coefficient of expression, a supervised learning approach was used to reduce the original 81-gene signature to a smaller 44-gene predictive signature having similar accuracy.

Using a validation set of 66 tumors, the 44-gene signature correctly classified 27 of 35 patients with progressive disease and 15 of 31 patients with objective response. Univariate analysis showed the response predictions by the 44-gene signature to be superior to predictions based on the analysis of traditional factors such as menopausal status, disease-free interval, first dominant site of relapse, estrogen and progesterone receptor status.

Univariate and multivariate analysis showed the 44-gene signature to be predictive of progression free survival, e.g., the time until tumor progression was seen.

### Individual Signature Genes

Expression levels of mRNA from individual genes in the 81-gene signature were measured by quantitative PCR as disclosed in the Examples below. The qPCR data was correlated with the mircroarray data. Of eight tested genes (CASP2, DLX2, USP9X, CHD6, MST4, RABEP, SIAH2, and TNC), Spearman rank correlations were positive for all except for MST4.

### Functional Clusters of Signature Genes

The genes in the 81-gene predictive signature contained 15 ESTs and 66 known genes. See the listing of genes in FIG. 2. Functional annotation of these genes showed clusters of genes involved with estrogen action, apoptosis, extracellular matrix formation, and immune response. Additional genes function in glycolysis, transcription regulation, and protease inhibition.

Seventeen genes were regulated by or associated with estrogen (receptor) action, with 9 genes upregulated (LOC51186; TSC22; TEMP3; SPARC; GABARAPL1; CFP1; LDHA; ENO2; Hs. 99743) and 8 genes downregulated (TXN2; CDC42BP4; HLA-C; PSME1; Hs. 437986; SIAH2; UGCG; FMNL) in the primary tumors of tamoxifen-resistant patients, as shown in FIG. 2.

Six genes associated with the extracellular matrix (TIMP 3, FN1, LOX, COL1A1, SPARC, AND TNC) and were overexpressed in patients with tamoxifen-resistant disease. Another cluster of seven genes were associated with apoptosis (IL4R, LDHA, MSP2K4, NPM1, SIAH2, CASP2, and TXN2), while two genes were related to anti-apoptosis activities (AP15, BNIP3). Four apoptosis genes were upregulated (AP15, NPM1, LDHA, BNIP3), while the other 5 were downregulated in primary tumors of patients with tamoxifen-resistant disease. A cluster of 4 genes linked to the immune system was downregulated (FCGRT, PSME-1, HLA-C, and NFATC3).

### Chromosome 17

The 81-gene signature contains a significant number of genes located on chromosome 17, and particularly localized to cytoband 17q21-q22. For example, 5 of 66 (6.5%) informative genes (APPBP2, COL1A1, EZH1, KIAA0563, and FMNL) are localized to this cytoband, as compared with 199 of 12771 known genes (1.3%) for the entire microarray.

### Tissue Samples

Breast cancer tissue samples useful for diagnostic assay can be obtained from primary tumor tissue, for example, biopsy tissue. In some instances, RNA may be obtained from the sample and used directly for analysis of expression. In general, RNA extracted from the tissue will be amplified, e.g., by polymerase chain reaction. For protein analysis, tissue can be paraffin-embedded and sectioned, for example, for immunohistochemstry and in situ hybridization analyses.

### Analysis of Gene Expression

In one embodiment, primary breast tumor tissue is analyzed for MRNA transcripts, for example, by hybridizing to cDNA probes. In another embodiment, the tissue is analyzed for protein, for example by immunoassay, for example, immunohisto chemistry. Individual genes of the 81-gene signature are known. NCBI Accession Numbers provided in FIG. 2 can be used to provide the nucleic acid and polypeptide sequences. Appropriate nucleic acid probes for hybridization and/or antibodies for immunoassay can be generated using known methods.

Gene expression in the primary tumor tissue sample is compared with the expression pattern of one or more marker genes identified from the 81-gene signature or from genes identified from cluster analysis and association with the genes of the 81-gene signature, as disclosed in the Examples below.

A nucleic acid marker as used herein is a nucleic acid molecule that, by its expression pattern in primary breast tumor tissue, alone or in combination with or compared with the expression patterns of one or more additional nucleic acid molecule, correlates with response or resistance to anti-estrogen, for example, tamoxiphen therapy for recurring breast cancer, or with outcome, such as progressive disease, stable disease, or progression-free survival.

Hybridization methods useful to analyze gene expression are well known. Nucleic acid molecules in the tumor tissue, for example MRNA, can hybridize under stringent hybridization conditions with a complementary nucleic acid probe. The nucleic acid hybridization probe need not be a full-length molecule, but can be a fragment or portion of the a fragment of the full-length cDNA, a variant thereof, a SNP, or iRNA. The probe can also be degenerate, or otherwise contain modifications such as nucleic acid additions, deletions, and substitutions. What is required is that the probe retain its ability to bind or hybridize with the sample nucleic acid molecule, in order to recognize the expressed product in the sample.

### Assay Methods

Marker gene expression can be analyzed by known assay methods, including mehtods for detecting expressed nucleic acid molecules, such as RNA and encoded polypeptides. Nucleic acid probes and polypeptide binding ligands useful in such methods, can be prepared by conventional methods or obtained commercially. Detection of expression can be direct or indirect, using know labels and detection methods.

For analysis of nucleic acid molecules, standard methods, for example, microarray technology and qRT-PCR can be used to identify patterns of nucleic acid expression in the sample tissue. Methods of microarray technology, including DNA chip technology, gene chip technology, solid phase nucleic acid array technology, multiplex PCR, nucleic-acid spotted fluidity cards, and the like, are known, and may be used to determine the expression patterns of nucleic acid molecules in a patient's tumor sample. In one embodiment, array of identified nucleic acid probes is provided on a substrate. In a preferred embodiment, the expression of signature genes is assayed by qPCR techniques.

For analysis of expressed polypeptides, known binding assay methods, such as immunoassay methods can be used. Examples include imunohitochemistry, ELISA, radioimmunoassay, BIACore, and the like.

### EXAMPLES

The invention is described herein with reference to the following examples that serve to illustrate the embodiments of the invention, and are not intended to limit the scope of the invention in any way.

### Example 1

### Identification of a Predictive Gene Signature

The Examples below describe studies undertaken to determine the measurable effect of anti-estrogen, for example, tamoxiphen therapy for breast cancer on tumor size and on time until tumor progression (progression free survival). The analysis was performed on 112 estrogen receptor positive primary breast cancer samples from patients who developed advanced disease that showed the most pronounced types of response (objective response versus progressive disease from the start of treatment). In addition, these studies describe underlying gene (signaling) pathways that provide novel potential targets for therapeutic intervention.

### METHODS

Patients and Treatment The study design was approved by the medical ethical committee of the Erasmus MC Rotterdam, the Netherlands (EC 02.953). To evaluate the predictive value of gene-expression profiling in relation to tamoxifen treatment in patients with recurrent breast cancer, 112 fresh frozen ER-α-positive (>=10 fmol/mg of protein) primary breast tumor tissue specimens of patients with primary operable (invasive) breast cancer diagnosed between 1981 and 1992 were included. The median age at time of primary surgery (breast conserving lumpectomy, 33 patients; modified mastectomy, 79 patients) was 60 years (range, 32-89 years).

In this retrospective study, all patients were selected for disease recurrence (14 local or regional relapse, 86 distant metastasis) that was treated with tamoxifen (40 mg daily) as first-line treatment. At the start of tamoxifen treatment, the median age was 63 years (range 33-90 years), and 27 patients (24%) were premenopausal. None of the patients had received endocrine (neo)adjuvant systemic therapy nor were exposed to any hormonal treatment at an earlier stage, i.e. hormo-naïve. Eighteen patients (16%) received adjuvant chemotherapy. Of these patients, 7 were postmenopausal whereas 11 were premenopausal at time of surgery. At start of tamoxifen monotherapy 8 patients were still pre-menopausal, whereas 3 patients changed to the post-menopausal status before recurrence. Two of these three patients showed objective response to tamoxifen. Therefore, chemocastration as prior endocrine therapy could not have had a significant impact on the results.

The median follow-up of patients alive was 94 months (range, 21-165 months) from primary surgery, and 53 months (range, 2-131) from the start of tamoxifen treatment. Tumor progression after tamoxifen occurred in 103 (92%) of the patients. During follow-up, 94 patients (84%) died. After tumor progression on first-line tamoxifen treatment 69 patients were treated with one or more additional endocrine agents, while 64 patients were subsequently treated with one or more regimens of chemotherapy such as cyclophosphamide methotrexate 5-Fluorouracil (CMF) or 5-fluorouracil, adriamycin, cyclophosphamide (FAC) after the occurrence of hormonal resistance.

Criteria for follow-up, type of response, response to therapy was defined by standard UICC criteria (Hayward, et al., 1977, Cancer, 39:1289-94), and for progression free survival Were described previously (Foekens, et al., 2001, Cancer Res., 61:5407-14). Complete and partial response (CR and PR) was observed in 12 and 40 patients, respectively, resulting in 52 patients with an objective response (OR); progressive disease (PD) within 3-6 months from start of treatment was observed in 60 patients. Median progression free survival-time of objective response was 17 months, whereas the median progression free survival-time of patients with progressive disease was 3 months.

### RNA Isolation, Amplification And Labeling

Total RNA was isolated from 30 μm frozen sections (approximately 20-50 mg tumor tissue) with RNABee (Campro Scientific). The percentage of tumor cells was determined in two Haematoxylin eosin stained frozen 5 μm sections that were cut before and after sectioning for RNA isolation. The tumor samples had a median tumor content of 65%. A T7dT oligo primer was used to synthesize double-stranded cDNA from 3 μg total RNA and subsequently to generate aRNA by in vitro transcription with T7 RNA polymerase (T7 MEGAscript™ High Yield Transcription kit, Ambion Ltd., Huntingdon, UK). Two micrograms of aRNA was labeled with Cy3 or Cy5 (CyDye, Amersham Biosciences) in a reverse transcription reaction. The labeled cDNA probes were purified using Qiagen PCR clean up columns (Qiagen, Westburg BV, Leusden, The Netherlands).

Similar to the Stanford protocol, a cell line pool of 13 cell lines derived from different tissue origins was used as reference for all microarray hybridizations (details are available at MIAMExpress (http://www.ebi.ac.uk/miamexpress/). Probes of the cell line pool were always labeled with Cy5.

### Quantitative Real-Time PCR

Total RNA isolated for the microarray analysis was used to verify the quantity of specific messengers by real-time PCR. The RNA was reverse-transcribed and real-time PCR products were generated in 35 cycles from 15 ng cDNA in an ABI Prism 7700 apparatus (Applied Biosystems, Foster City, USA) in a mixture containing SYBR-green (Applied Biosystems, Stratagene) and 330 nM primers for differentially expressed genes (i.e. CASP2, DLX2, EZH1, CHD6, MST4, RABEP, SIAH2, and TNC). SYBR-green fluorescent signals were used to generate Cycle threshold (Ct) values from which MRNA ratios were calculated when normalized against the average of three housekeeping genes, i.e. hypoxanthine-guanine phospho-ribosyltransferase (HPRT), porphobilinogen deaminase (PBGD), and β-2-microglobulin (B2M) (Martens, et al., 2003, Thromb. Haemost., 89:393-404).

cDNA Microarrays: Preparation, Hybridization, and Data Acquisition

Microarray slides were manufactured at the Central Microarray Facility at the Netherlands Cancer Institute (Weige, et al., 2003, Proc. Natl. Acad. Sci. U.S.A., 100:15901-5). Sequence-verified clones obtained from Research Genetics (Huntsville, Ala.) were spotted with a complexity of 19,200 spots per glass slide using the Microgrid II arrayer (Biorobotic, Cambridge, U.K.) The gene ID list can be found at http://microarrays.nki.nl. Labeled cDNA probes were heated at 95° C. for 2 minutes and added to preheated hybridization buffer (Slide hybrization buffer 1, Ambion). The probe mixture was hybridized to cDNA microarrays for 16 hours at 45° C.

Fluorescent images of microarrays were obtained by using the GeneTAC™ LS II microarray scanner (Genomic Solutions; Perkin Elmer). IMAGENE v5.5 (Biodiscovery, Marina Del Rey, Calif.) was used to quantify and correct Cy3 and Cy5 intensities for background noise. Spot quality was assessed with the flagging tool of IMAGENE, in this study set at R>2 for both Cy3 and Cy5. Fluorescent intensities of each microarray were normalized per subgrid using the NKI MicroArray Normalization Tools (http://dexter.nki.nl) to adjust for a variety of biases that affect intensity measurements (e.g. color-, print tips, local background bias) (Yang, et al., 2002, Nucleic Acids Res., 30:e15). All ratios were log2 transformed.

### Data Analysis and Statistics

Microarray data analyses were performed with the software packages BRB Array Tools, developed by the Biometric Research Branch of the US National Cancer Institute, (http://linus.nci.nih.gov/BRB-ArrayTools.html), and Spotfire (www.spotfire.com, Goteborg, Sweden and Sommerville, Mass.). BRB was implemented for statistical analysis of microarray data whereas Spotfire was used for cluster analysis. The class comparison tool in BRB combines a univariate F-test and permutation test (n=2000) in order to find discriminating genes and to confirm their statistical significance. In the class comparison a significance level of 0.05 was chosen in order to limit the number of false negatives.

Spotfire was used to perform hierarchical clustering. To analyze microarray data from different batches of slides, genes were Z-score normalized per batch. The Z-score was defined as [value—mean]/SD. After normalization, microarray data were clustered via complete linkage. The similarity measure for clustering was based on cosine correlation and average value.

Sensitivity, specificity, positive and negative predictive value (PPV and NPV, respectively) and odds ratios (OR) were calculated and presented with their 95% confidence interval (CI). The data are shown in Table 2. The performance of the signature in the validation set was determined via the likelihood ratio of the Chi square test. A supervised learning approach was applied to reduce the 81 differentially expressed genes to a smaller 44-gene predictive signature. First, all 81 genes were rank ordered on the basis of their significance as calculated with the BRB class comparison tool. Next, starting with the most significant gene, the Pearson correlation coefficient of expression with the other 80 genes was calculated. Succeeding genes were excluded from the signature as long as their expression correlated significantly (P<0.05) with the most significant gene. The first gene of the 81 gene profile that did not correlate with expression of the most significant gene was added to the final signature, and the whole procedure of expression correlation analysis with this second gene was repeated with the remaining less significant genes. In this way, genes with overlap in their expression were removed and the 44-gene predictive signature was derived.

The predictive score for the traditional-based model included menopausal status, disease free interval (DFI>12 months versus DFI≦12 months after primary surgery), dominant site of relapse (relapse to viscera or bone versus relapse to soft tissue), log estrogen receptor (ER) and log progesterone receptor (PgR) levels. In the analyses of progression free survival, the Cox proportional hazards model was used to calculate the hazard ratios (HRs) and 95% CI. Survival curves were generated using the method of Kaplan and Meier (1958, J. Am. Stat. Assoc., 53:457-481) and a log rank test for trend was used to test for differences. Correlation between microarray data and real-time PCR data was determined with Spearman rank correlation test. Computations were performed with the STATA statistical package, release 7.0 (STATA Corp., College Station, Tex.). All p-values are two-sided.

### Method of Classification

For the validation of the 44-gene signature, a classification algorithm (Gene Prediction Tool (GPT)) was developed that is comparable to the Compound Covariate Predictor (CCP) from BRB Array Tools. In detail, GPT applies two cut-off values instead of the midpoint used in the CCP tool for classification. The two thresholds are the median values of progressive disease and objective response and are defined in the tumors of the training set.

To obtain a robust classification algorithm, genes from the signature only become classifiers whenever the expression values are outside the two thresholds and as a result mainly represent one class, either progressive disease or objective response. When the expression level falls between the two cut-off values, the gene is excluded as classifier because the value can represent both response classes, i.e. progressive disease but also objective response. The gene classifiers from the predictive 44-gene signature are identified for each tumor from the validation set using the algorithms described herein. Finally, the ratio between the identified response predicting genes and resistance predicting genes determines the predicted signature-based response outcome.

### Mathematical Algorithm For Gene Prediction Tool

| | |
|---|---|
| Threshold Objective Response for gene x: | Kx = MEDIAN (Mx:AKx) |
| Threshold Progressive disease for gene x: | Jx = MEDIAN(ALx:BFx) |
| Classification Constant for gene x: | Lx = IF(Kx >= Jx, 1, −1) |
| Constant for gene x: | either 1 for response or −1 for |
| | resistance |
| Gene x Tumor Classification: | My = $Lx * IF(Mx > |
| | MAX($Jx, $Kx), 1, |
| | IF(MIN($Jx, $Kx), −1, 0)) |
| Tumor classification for gene x: | either 1, −1, or 0 (= not |
| | informative) |

*Operation as performed on Excel spreadsheet

### Results

### Selection Of Differentially Expressed Genes and Predictive Signature

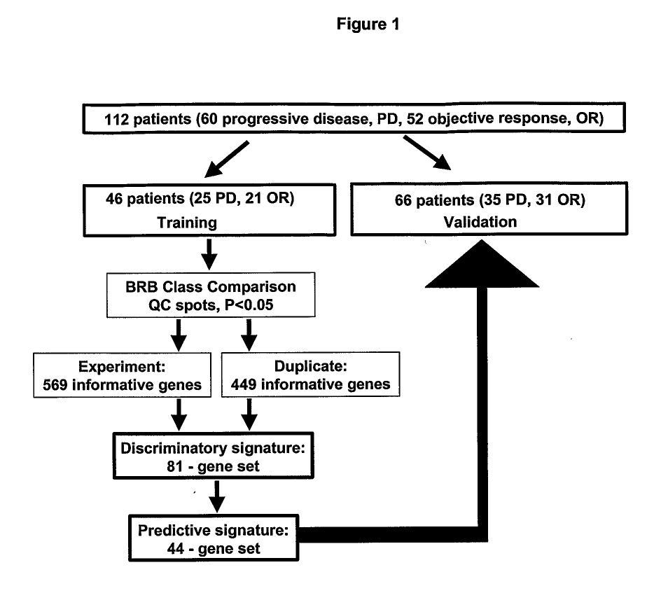

To select discriminatory genes for the type of response to tamoxifen, a training set of 46 tumors was defined that comprised primary tumors of 25 patients with progressive disease (PD) and of 21 patients with objective response (OR, see FIG. 1). The tumor RNAs of this training set were hybridized, in duplicate, and genes/ESTs that had less than 90% present calls over the experiments were eliminated. This resulted in 8555 and 7087 evaluable spots, respectively. Using a significance level of 0.05 in the BRB class comparison tool, 569 and 449 genes, respectively, were differentially expressed between the progressive disease and objective response subsets. The overlap, i.e. 81 genes, was designated as the differentially expressed signature.

After supervised hierarchical clustering (shown in FIG. 2), this discriminatory signature correctly classified 21 of 25 patients with progressive disease (84% sensitivity; 95% CI: 0.63-0.95) and 19 of 21 patients with objective response (91% specificity, 95% CI 0.68-0.98) with an odds-ratio of 49.8 (p<0.0001). The positive predictive value and negative predictive value for resistance to tamoxifen were 91% and 83%, respectively. Further analysis, rank-ordering of genes on the basis of significance level, followed by a step-up calculation of correlation coefficient of expression, reduced the initial set of 81 genes to a smaller 44-gene predictive signature with similar accuracy.

### Example 2

### Validation of Predictive Gene Signature: Correlation to Clinical Response and Time to Treatment Failure

### Type of Response

In a validation set of 66 tumors, the predictive 44-gene signature correctly classified 27 of 35 patients with progressive disease (77% sensitivity, 95% CI: 0.59-0.89) and 15 of 31 patients with objective response (48% specificity, 95% CI: 0.31-0.67) with an odds ratio of 3.16 (95% CI: 1.10-9.11, p=0.03). In univariate analysis for response, the predictive signature appeared to be superior, i.e. more than 2-fold higher odds ratio, to most traditional factors (i.e. menopausal status, disease-free interval, first dominant site of relapse, estrogen and progesterone receptor status), of which only estrogen receptor-level (odds ratio, 1.54; 95% CI: 1.00-2.40; p=0.05) and progesterone receptor-level (odds ratio, 1.37; 95% CI: 1.05-1.79; p=0.02) showed significant predictive value. In multivariate analysis for response, the signature-based classification did not significantly (increase in X²=1.45) add to the traditional based-factor score (data not shown).

### Progression Free Survival

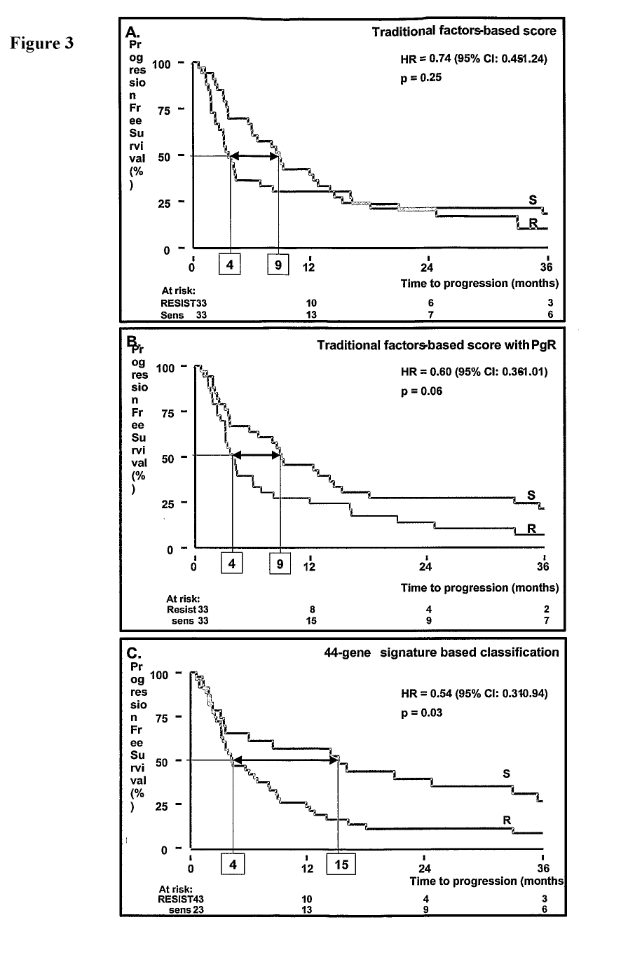

In addition, in univariate analysis only the 44-gene signature (hazard ratio, 0.54 [95% CI: 0.31-0.94]; p=0.03) and progesterone receptor-level (hazard ratio, 0.83 [95% CI: 0.73-0.96]; p=0.01) were significantly correlated with a longer time until tumor progression and this was retained for the signature in the multivariable analysis (hazard ratio, 0.48 [95% CI: 0.26-0.91]; p=0.03). Progesterone receptor is also independent, but with a less striking hazard ratio (0.82 [95% CI: 0.71-0.94]; p=0.01). After addition of the signature-based classification to the traditional-factors-based score the increase in ² was 5.18 (df=1, p=0.02), indicating that the predictive signature independently contributed to the traditional predictive factors for progression free survival. In Kaplan-Meier analyses, the median difference in progression free survival time for patients with a favorable and poor response was 2-fold longer when the 44-gene signature (FIG. 3c) was used in comparison to the traditional factors-based score without (FIG. 3a) and with PgR (FIG. 3b) (i.e. 11 months versus 5 months, see FIG. 3).

TABLE 1
Univariable and multivariable analysis for PFS after start of
tamoxifen treatment in the validation set of 66 patients with
advanced breast cancer.

| | Univariable (N = 66) | | | Multivariable (N = 66) | | |
|---|---|---|---|---|---|---|
| | HR | [95% CI] | p | HR | [95% CI] | p |
| Traditional | | | | | | |
| factors: | | | | | | |
| Menopausal | 1.07 | [0.57-2.00] | 0.83 | 1.16 | [0.58-2.33] | 0.67 |
| status^{a} | | | | | | |
| Dominant site | | | | | | |
| of relapse: | | | | | | |
| Bone to soft | 1.56 | [0.70-3.47] | 0.28 | 1.80 | [0.76-4.26] | 0.19 |
| tissue | | | | | | |
| Viscera to | 1.26 | [0.47-2.79] | 0.57 | 1.42 | [0.60-3.34] | 0.42 |
| soft tissue | | | | | | |
| Disease Free | 0.92 | [0.53-1.57] | 0.75 | 1.08 | [0.61-1.90] | 0.80 |
| Interval^{b} | | | | | | |
| Log ER | 0.83 | [0.66-1.06] | 0.13 | 0.88 | [0.68-1.14] | 0.33 |
| Log PgR | 0.83 | [0.73-0.96] | 0.01 | 0.82 | [0.71-0.94] | 0.01 |
| Microarray | | | | | | |
| 44-gene | 0.54 | [0.31-0.94] | 0.03 | 0.48 | [0.26-0.91] | 0.03 |
| signature^{c} | | | | | | |

^{a}Menopausal status: post- vs premenopausal;
^{b}DFI: >12 months vs ≦12 months;
^{c}44-gene signature: sensitive vs resistant.

### Example 3

### Independent Confirmation Of Gene-Expression

The mRNA expression levels of 8 genes of the 81-gene signature were analyzed by quantitative real-time PCR. The genes included: CASP2, DLX2, USP9X, CHD6, MST4, RABEP, SIAH2, and TNC. qPCR data was correlated with microarray data. Spearman rank correlations were positive for all genes but MST4.

### Example 4

### Functional Analysis of the 81-Gene Predictive Signature

The 81-gene signature described above in Example 1 was analyzed for the functional aspects of the genes contained in the signature. The genes were examined for functional relationships using Ingenuity Pathway Analysis tools. (Mountain View, Calif.) The signature contains 15 ESTs and 66 known genes (see FIG. 2). Functional annotation of the genes in the signature showed genes involved in estrogen action (26%), apoptosis (14%), extracellular matrix formation (9%), and immune response (6%). The remaining genes function in glycolysis, transcription regulation, and protease inhibition.

The patterns of expression of many genes that are associated with anti-estrogen, for example, tamoxiphen resistance and sensitivity are highly complex. The 81 differentially expressed genes includes, as expected, genes regulated by or associated with estrogen (receptor) action (van 't Veer, et al., 2002, Nature, 415:530-6; Tang, et al., 2004, Nucleic Acids Res., 32 Database issue: D533-6; Pusztai, et al., 2003, Clin. Cancer Res., 9:2406-15; Gruvberger, et al., 2001, Cancer Res., 61:5979-84; Charpentier et al., 2000, Cancer Res., 60:5977-83; Frasor, et al. 2003, Endocrinology, 144:4562-74), but also genes involved in extracellular matrix formation and apoptosis.

Seventeen genes were regulated by or associated with estrogen (receptor) action, of which 9 genes showed upregulation (LOC51186; TSC22; TIMP3; SPARC; GABARAPL1; CFP1; LDHA; ENO2; Hs. 99743) and 8 genes downregulation (TXN2; CDC42BP4; HLA-C; PSME1; Hs. 437986; SIAH2; UGCG; FMNL) in the primary tumors of patients who were resistant to tamoxifen therapy for recurring breast cancer (see FIG. 2). Several of these estrogen (co-)regulated genes (LDHA, TXN2, and SIAH2) have been linked to apoptosis.

A cluster of 6 genes was identified as associated with the extracellular matrix (ECM). These genes, TIMP3, FN1, LOX, COL1A1, SPARC, and TNC were overexpressed in the primary tumors of patients that demonstrated resistance to anti-estrogen, for example, tamoxiphen therapy for treatment of recurring breast cancer (progressive disease).

Besides cytostatic effects, the anti-estrogen tamoxifen is known to have cytolytic effects by induction of apoptosis, as reviewed by Mandlekar and Kong (Mandlekar, et al. 2001, Apoptosis, 6:469-77). Based on Swiss prot, PubMed, and Ingenuity analysis information, nine genes (LOC51186; TSC22; TIMP3; SPARC; GABARAPL1; CFP1; LDHA; ENO2; Hs. 99743) in the 81-gene signature are related to programmed cell death, of which three genes inhibit apoptosis (API5, NPM1, and TXN2) whereas three other genes induced apoptosis (CASP2, MAP2K4, and SIAH2). Interestingly, the two latter genes (MAP2K4, SIAH2) induce the apoptotic machinery of fibroblasts.

Seven genes of the Signature were associated with apoptosis (IL4R, LDHA, MAP2K4, NPM1, SIAH2, CASP2, and TXN2), whereas two other signature genes (API5, BNIP3) were related to anti-apoptosis processes.

In general, the expression patterns indicate that anti-estrogen, for example, tamoxiphen resistance is mainly associated with inhibition of apoptosis. Interestingly, 4 apoptosis genes (API5, NPM1, LDHA, and BNIP3) were upregulated and 5 genes (IL4R, MAP2K4, SIAH2, CASP2, and TXN2) were downregulated in primary tumors of patients that were resistant to anti-estrogen, for example, tamoxiphen therapy for treatment of recurring breast cancer (progressive disease).

### Example 5

### Specifc Set of Useful Marker Genes

Ten genes selected from the 81-gene signature (CHD6, FN1, TNC, CASP2, EZH1, RABEP1, THRAP2, SIAH2, DEME-6, COX6C) were analyzed to date against 272 tumors. Uni- and multivariable analyses were performed to determine the response and duration of response (progression free survival), using the methods described above. In multivariable analysis the individual genes were compared with the clinically used model of traditional predictive factors (i.e., menopausal status, disease-free survival, dominant site of relapse, log ER, and log PR).

Specific calculation of threshold values (cutpoints) (Table 3) for prediction of Overall Response and Progression Free Survival were calculated as described above. As shown in Tables 3-6, marker genes DEME-6, CASP2, and SIAH2 were useful as individual markers of clinical outcome. Reliability of the prediction increased with combinations of the markers (see Tables 5 and 6).

None of the ECM genes in the signature is found in the 70-gene classifier for poor prognosis of node negative breast cancer patients by van 't Veer et al. (van 't Veer, et al., 2002, Nature, 415:530-6), suggesting that the ECM gene cluster is specific for the prediction of tamoxifen resistance. Furthermore, SPARC/osteonectin, a myoepithelial cell marker that is estrogen (co-)regulated, was recently described as an independent marker of poor prognosis in unselected breast cancers (Iacobuzio-Donahue, et al., 2002, Cancer Res., 62:5351-7; Mackay, et al., 2003, Oncogene, 22:2680-8; Jones, et al., 2004, Cancer Res., 64:3037-3045). In addition, a new cluster of genes linked to the immune system (FCGRT, PSME1, HLA-C, and NFATC3) was downregulated in the patients with progressive disease compared to those with objective response.

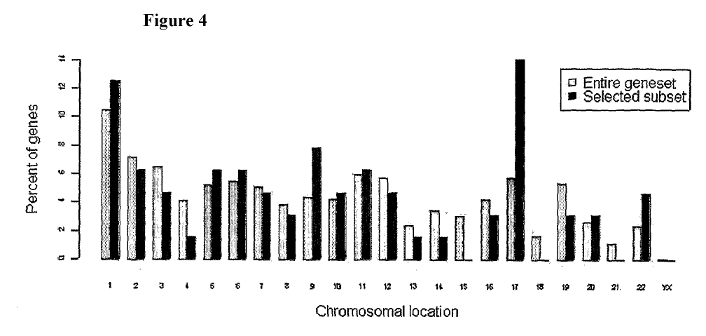

The 81-gene signature showed an overrepresentation of genes located to chromosome 17, but an under representation of genes located to chromosomes 4, 15, 18 and 21 (FIG. 4). Genes localized to cytoband 17q21-q22 appeared to be significantly (p=0.03) overrepresented, i.e. 5 of 66 informative genes (i.e. APPBP2, COL1A1, EZH1, KIAA0563 and FMNL) in the signature (6.5%) compared to 199 of 12771 known genes (1.5%) for the whole microarray.

### DISCUSSION

The studies described above in Examples 1-4 demonstrate that expression array technology can be effectively and reproducibly used to classify primary breast cancer tumors according to a predicted resistance or sensitivity to anti-estrogen, for example, tamoxifen treatment for recurring breast cancer. An 81-gene signature with multiple individual genes predictive of response and outcome, alone or in combination with other genes is described and validated. A 44-gene signature is described that predicted anti-estrogen, for example, tamoxiphen therapy outcome in 112 breast cancer patients with ER positive recurrent disease. Overall, a prediction of anti-estrogen, for example, tamoxiphen resistance was accomplished with an accuracy of 80%. Moreover, the 44-gene gene signature predicted a significantly longer progression free survival time that is superior to the prediction obtained by a traditional factors-based score. Differences in RNA expression were confirmed by quantitative real-time PCR.

The predictive value of the 44-gene signature compares favorably and contributes independently with that of traditional prognostic factors, including the estrogen receptor, currently the validated factor for response prediction to hormonal therapy in breast cancer. The estrogen receptor, present in about 70-75% of breast cancers, correctly predicts response to tamoxifen in about 50-60% of the patients (Osborne, 1998, N. Engl. J Med. 339:1609-18), while the gene signature predicts resistance to tamoxifen in 77% of the patients in the validation set.

The present 44-gene signature, due to its significant association with time to treatment failure, may be used to classify patients based on time to treatment failure.

In general, the arrays used in these different studies comprise different genes/ESTs than those disclosed in the prior art. Of these arrays, approximately half of the genes show overlap. This could result in few overlapping genes in the generated gene-signatures. Therefore, comparison of pathways based on the extracted gene signatures from different studies could be more informative. At present, none of these differentially expressed genes that are regulated by or associated with estrogen (receptor) action have been directly linked by others with endocrine resistance in clinical samples. The data described herein provides a better understanding of endocrine resistance and provides novel potential therapeutic targets for individualized treatment.

A diagnostic assay was recently developed by Genomic Health, the Oncotype DX diagnostic assay based on a candidate gene selection (not genome wide) approach. This test provides a recurrence score for lymph node negative breast cancer patients with estrogen receptor positive tumors that have received adjuvant tamoxifen (Paik, et al., 2003, Breast Cancer Res. Treat., 82:S10). Their multiplex 21-gene test includes genes associated with proliferation, estrogen and HER2 action, invasion and 5 control genes. None of the genes however, overlap with the 81-gene signature that was selected through microarray based gene expression profiling.

Recently, Sgroi et al. (Ma, et al., 2004, Cancer Cell, 5:607-16) also analyzed tumors from patients with adjuvant tamoxifen therapy using microarray analyses. They extracted a two-gene ratio that predicts “a tumor's response to tamoxifen or its intrinsic aggressiveness, or both”. Interestingly Sgroi et al. (Ma, et al., 2004, Cancer Cell, 5:607-16) showed that HOXB13, located to 17q21 was overexpressed in tamoxifen resistant cases with recurrence after adjuvant tamoxifen. In the 81-gene signature, we observed 5 genes located to chromosome 17q21-22 that could be of importance for tamoxifen resistance. In this region, the signature gene COL1A1 was discriminative and highly expressed in the signature. Moreover, HOXB13, like COL1A1, is not positioned in the 17q21 HER2/ERBB2 amplicon (Hyman, et al., 2002, Cancer Res., 62:6240-5) but in the second of three regions (i.e. 17q12-HER2-, 17q21.2-HOXB2-7-, 17q23-PPM1D-) highly amplified in breast cancer. This implies that genes other than those of the ERBB2 amplicon region, like HOXB13 and COL1A1 are important for resistance to tamoxifen and present potential therapeutic targets.

The expression of the other 4 signature genes located to chromosome 17q does not correlate with the ERBB2 expression, since they (EZH1, FMNL, KIAA0563, and APPBP2) were down regulated in the tamoxifen resistant tumors. This region has been implicated for LOH in 30% of breast cancer cases (Osborne, et al., 2000, Cancer Res., 60:3706-12). Only recently, JUP/plakoglobin/gamma-catenin was identified as a LOH, whereas LOH of BRCA1 is frequently observed in high-grade tumors (Ding, et al., 2004, Br. J Cancer, 90:1995-2001). The signature gene EZH1 located between JUP and BRCA1 may, therefore, be another LOH candidate gene.

Numerous reports have described that ERBB2 amplification and over-expression in ER positive patients is associated with a reduction in response rate to first-line hormone therapy (Lipton, et al., 2003, J. Clin. Oncol., 21:1967-72; Ferrero-Pous, et al., 2000, Clin. Cancer Res., 6:4745-54; Wright, et al., 1989, Cancer Res., 49:2087-90). Since the expression patterns of the 5 signature genes on 17q21-q22 are not significantly correlated with ERBB2 expression in this array study, this indicates that another, yet unknown, mechanism may be activated.

An 81-gene signature of differentially expressed genes and a 44-gene signature that predicts anti-estrogen, for example, tamoxifen therapy resistance and time to progression in ER-positive breast cancer patients with recurrent disease have been developed. The gene signatures demonstrate a significantly better performance than the commonly used traditional clinical predictive factors in uni- and multivariate analyses, and (3). In contrast to the traditional factors site of relapse and disease free interval (DFI), the prediction of response can be derived from the gene-expression profile of primary tumors.

### Objective Response, Stable Disease, and Progressive Disease

The 81-gene signature was validated with quantitative PCR analysis (as described above) on RNA obtained from a larger series of 272 tumors from breast cancer patients who underwent first-line tamoxifen therapy for advanced disease. Included were patients having stable disease. Of these, 59 showed an objective response, 120 had stable disease, and 93 had progressive disease.

Ten genes selected from the 81-gene signature (CHD6, FN1, TNC, CASP2, EZH1, RABEP1, THRAP2, SIAH2, DEME-6, COX6C) have been analyzed to date against all 272 tumors. Uni- and multivariable analyses have been performed to determine the response and duration of response (progression free survival). In multivariable analysis the individual genes were compared with the clinically used model of traditional predictive factors (i.e., menopausal status, disease-free survival, dominant site of relapse, log ER, and log PR).

Clinical implications for patients predicted to have a poor response to tamoxifen therapy are that these patients should be candidates for other treatments or novel therapies, based on different targets present in their tumor profiles. This will reduce the use of ineffective treatments.

TABLE 2

| Clust. No. | spot | nki-id | Acc. code | Unigene | Gene_Symbol | Location | 44 sign. | gene |
|---|---|---|---|---|---|---|---|---|
| 1 | 4661 | 28241 | AA035436 | Hs.227913 | API5 | 11p12-q12 | | API5L1 |
| 2 | 2613 | 10578 | H96654 | Hs.15984 | LOC51186 | Xq22.1 | | LOC51186 |
| 3 | 1486 | 36330 | AI359120 | Hs.45207 | CHD6-pending | 20q12 | | CHD6 |
| 4 | 6665 | 28042 | AA398237 | Hs.114360 | TSC22 | 13q14 | 6 | TSC22 |
| 5 | 4155 | 29639 | AA206591 | Hs.169514 | | 1 | | |
| 6 | 2662 | 27978 | AA479202 | Hs.245188 | TIMP3 | 22q12.3 | 25 | TIMP3 |
| 7 | 13600 | 1130 | R62612 | Hs.287820 | FN1 | 2q34 | 28 | FN1 |
| 8 | 7435 | 7366 | W70343 | Hs.102267 | LOX | 5q23.2 | | LOX |
| 9 | 2579 | 23688 | R48844 | Hs.172928 | COL1A1 | 17q21.3-q22.1 | | COL1A1 |
| 10 | 11500 | 8241 | H95960 | Hs.111779 | SPARC | 5q31.3-q32 | | SPARC |
| 11 | 2239 | 9654 | T55569 | Hs.9911 | FLJ11773 | 12q13.13 | | FLJ11773 |
| 12 | 6181 | 23441 | H85107 | Hs.222581 | | 11 | 10 | |
| 13 | 8290 | 27874 | AA598955 | Hs.289114 | TNC | 9q33 | | HXB |
| 14 | 679 | 31446 | AI266693 | Hs.144058 | EBSP | 17q25.2 | 36 | DKFZP564C103 |
| 15 | 14076 | 29372 | AA454540 | Hs.356786 | GNAQ | 9q21.2 | | GNAQ |
| 16 | 5749 | 22769 | AA644587 | Hs.172694 | LOC117584 | 17q12 | 40 | |
| 17 | 11338 | 6753 | AA137072 | Hs.294141 | SMARCA4 | 19p13.3 | 26 | |
| 18 | 15827 | 5156 | AA446839 | AA446839 | BNIP3 | 10q26.3 | 22 | BNIP3 |
| 19 | 8522 | 25666 | AA933888 | Hs.7956 | | 21 | 19 | |
| 20 | 11771 | 4569 | N95358 | Hs.121576 | MYO1B | 2q12-q34 | | MYO1B |
| 21 | 11479 | 31155 | H01495 | Hs.4147 | TRAM | 8q13.1 | | TRAM |
| 22 | 10259 | 11097 | T60160 | Hs.336429 | GABARAPL1 | 12p13.31 | | GABARAPL1 |
| 23 | 9248 | 29464 | AA489638 | Hs.165998 | PAI-RBP1 | 1p31-p22 | | PAI-RBP1 |
| 24 | 9770 | 7617 | H72683 | Hs.6820 | CFP1 | 10p11.21 | 38 | TIMM10 |
| 25 | 1095 | 8322 | AA669758 | Hs.355719 | NPM1 | 5q35 | | NPM1 |
| 26 | 11106 | 9261 | AA668425 | Hs.904 | AGL | 1p21 | | AGL |
| 27 | 7318 | 10888 | AA431187 | Hs.429780 | | 12 | 1 | |
| 28 | 17976 | 1609 | AA497029 | Hs.2795 | LDHA | 11p15.4 | | LDHA |
| 29 | 277 | 31206 | AA625960 | Hs.208414 | MCFP | 7q21.12 | 3 | |
| 30 | 7080 | 31468 | AI340932 | Hs.109590 | GENX-3414 | 4q24-q25 | 33 | GENX-3414 |
| 31 | 18213 | 4897 | H71881 | Hs.395779 | CAMTA1 | 1p36.23 | 41 | KIAA0833 |
| 32 | 16231 | 7345 | W37375 | Hs.433540 | DNAJC8 | 1p35.2 | | DNAJC8 |
| 33 | 3824 | 5453 | N72215 | Hs.406455 | PSAP | 10q21-q22 | 16 | PSAP |
| 34 | 4309 | 9425 | AA427899 | Hs.179661 | OK/SW-cl.56 | 6p21.33 | 18 | FKBP1A |
| 35 | 9888 | 28875 | AI083527 | Hs.146580 | ENO2 | 12p13 | 35 | |
| 36 | 7271 | 20608 | AA504120 | Hs.99743 | | 14 | 20 | |
| 37 | 11498 | 8193 | AA663983 | Hs.83848 | TPI1 | 12p13 | | TPI1 |
| 38 | 15504 | 9014 | AA176957 | Hs.83870 | NEB | 2q22 | 15 | NEB |
| 39 | 17856 | 31945 | AI669875 | Hs.95260 | FAM8A1 | 6p22-p23 | | FAM8A1 |
| 40 | 9894 | 8283 | AA677388 | Hs.2777 | ITIH1 | 3p21.2-p21.1 | | ITIH1 |
| 41 | 15976 | 1814 | AA046411 | Hs.84084 | APPBP2 | 17q21-q23 | | APPBP2 |
| 42 | 18934 | 4741 | H63760 | Hs.8037 | TM4SF9 | 4q23 | 44 | |
| 43 | 14373 | 1838 | N68825 | Hs.57730 | KIAA0133 | 1q42.13 | | KIAA0133 |
| 44 | 11798 | 10107 | H29308 | Hs.27804 | TUWD12 | 12q21.33 | 31 | |
| 45 | 3573 | 1637 | AA279072 | Hs.75339 | INPPL1 | 11q23 | 9 | [symbol] |
| 46 | 12954 | 29600 | AA481283 | Hs.108131 | CASP2 | 7q34-q35 | 11 | [symbol] |
| 47 | 12116 | 7485 | AA002091 | AA002091 | CACH-1 | 5q14.1 | 43 | |
| 48 | 15116 | 15344 | AA418826 | Hs.334690 | | 19q13.43 | | |
| 49 | 11829 | 10179 | AA446193 | Hs.405898 | KIAA0999 | 11q23.3 | 4 | KIAA0999 |
| 50 | 14590 | 18842 | N67797 | Hs.118194 | DBR1 | 3q22.3 | | DBR1 |
| 51 | 9484 | 21214 | AA679940 | Hs.211929 | TXN2 | 22q13.1 | 30 | TXN2 |
| 52 | 16944 | 29749 | AA703184 | Hs.194669 | EZH1 | 17q21.1-q21.3 | 24 | EZH1 |
| 53 | 11576 | 1611 | AA486275 | Hs.183583 | SERPINB1 | 6p25 | 2 | SERPINB1 |
| 54 | 12303 | 8991 | AA479888 | Hs.250535 | RAB5EP | 17p13.2 | | RABEP1 |
| 55 | 3784 | 6413 | R92446 | R92446 | | | | |
| 56 | 9322 | 25678 | AA975556 | Hs.347130 | FLJ22709 | 19p13.11 | 5 | FLJ22709 |
| 57 | 11069 | 28623 | AI364298 | Hs.13339 | PRPSAP2 | 17p11.2-p12 | 29 | PRPSAP2 |
| 58 | 5254 | 16253 | AA449773 | Hs.3903 | CDC42EP4 | 17q24-q25 | 34 | CEP4 |
| 59 | 13235 | 15896 | AA496000 | Hs.4084 | [symbol] | 12q24.22 | 23 | KIAA1025 |
| 60 | 12376 | 1623 | AA293365 | Hs.75217 | MAP2K4 | 17p11.2 | 39 | MAP2K4 |
| 61 | 9176 | 1624 | AA293306 | Hs.75545 | IL4R | 16p11.2-12.1 | 14 | IL4R |
| 62 | 18895 | 20125 | AA775791 | Hs.76662 | APH2 | 10q24.1 | 17 | MGC2993 |
| 63 | 7256 | 12952 | W68711 | Hs.170226 | FLJ38045 | 9q33.1 | 8 | |
| 64 | 1481 | 27768 | H59048 | Hs.172674 | NFATC3 | 16q22.2 | 21 | NFATC3 |
| 65 | 5493 | 8357 | AA464246 | Hs.277477 | HLA-C | 6p21.3 | 32 | HLA-C |
| 66 | 5856 | 31961 | AI676033 | Hs.301904 | FLJ12671 | 1q21.3 | 42 | FLJ12671 |
| 67 | 13177 | 1640 | T47815 | Hs.75348 | PSME1 | 14q11.2 | | PSME1 |
| 68 | 1136 | 8730 | 30668\|AI732 | Hs.111903 | FCGRT | 19q13.3 | | FCGRT |
| 69 | 9287 | 21376 | AA137228 | Hs.145599 | MLKN1 | 7q32 | 37 | |
| 70 | 4074 | 20591 | AA489015 | Hs.40919 | ALG2 | 9q22.33 | 7 | FLJ14511 |
| 71 | 14612 | 18320 | AA054643 | Hs.391828 | PARD6B | 20q13.12 | 13 | |
| 72 | 227 | 3036 | AA029042 | Hs.20191 | SIAH2 | 3q25 | | SIAH2 |
| 73 | 11367 | 11667 | N22323 | Hs.23643 | MST4 | Xq26.1 | | MST4 |
| 74 | 8167 | 11668 | AA165628 | Hs.432605 | UGCG | 9q31 | 27 | |
| 75 | 2719 | 20994 | AA912071 | Hs.432137 | DLX2 | 2q32 | | DLX2 |
| 76 | 18683 | 20995 | AA886199 | Hs.125783 | DEME-6 | 1p32.3 | | KIAA0452 |
| 77 | 13790 | 18662 | H85475 | Hs.339808 | KIAA0563 | 17q21.31 | | FLJ10120 |
| 78 | 12573 | 19023 | N51614 | Hs.100217 | FMNL | 17q21 | 12 | FMNL |
| 79 | 4373 | 1649 | T57841 | Hs.199402 | UFD1L | 22q11.21 | | UFD1L |
| 80 | 14752 | 2378 | AA456931 | Hs.351875 | COX6C | 8q22-q23 | | COX6C |
| 81 | 4550 | 23081 | AA626362 | Hs.116160 | WFDC6 | 20q13.12 | | |

Other Markers

| | | | |
|---|---|---|---|
| 101 | NM004456 | EZH2 | 7q35 |
| 102 | XM042066 | MAP3K1 | 5q11.2 |
| 103 | NM139049 | MAPK8 (JNK) | 10q11.22 |
| 104 | NM006311 | NCOR1 | 17p11.2 |
| 105 | NM012340 | NFATC2 | 20q13.2-q13.3(1) |
| 106 | NM004554 | NFATC4 | 14q11.2 |
| 107 | NM021724 | NRID1 | 17q11.2 |
| 108 | NM003620 | PPM1D | 17q23.1/2 |
| 109 | NM003031 | SIAH1 | 16q12 |
| 110 | NM004652 | USP9X | Xp11.4 |
| 111 | NM005428 | VAV1 | 19p13.3 |
| 112 | NM006113 | VAV3 | 1p22.3-p11/1p13.3 |

| | | | | Univariate analysis | | | | | | | |
|---|---|---|---|---|---|---|---|---|---|---|---|
| | | Fragment | | response | | | | | PFS | | |
| Clust. No. | qPCR | (bp) | N | OR | P | 95% CI | | HR | P | 95% CI | |
| 1 | Val | | | | | | | | | | |
| 2 | | | | | | | | | | | |
| 3 | Val264 | +/−95 | 246 | 1.408 | 0.067 | 0.98 | 2.03 | 0.992 | 0.923 | 0.84 | 1.18 |
| 4 | | | | | | | | | | | |
| 5 | | | | | | | | | | | |
| 6 | | | | | | | | | | | |
| 7 | Val264 | ? | 241 | 0.697 | 0.039 | 0.50 | 0.98 | 1.076 | 0.423 | 0.90 | 1.29 |
| 8 | | | | | | | | | | | |
| 9 | | | | | | | | | | | |
| 10 | | | | | | | | | | | |
| 11 | Val96 | 210 | | | | | | | | | |
| 12 | | | | | | | | | | | |
| 13 | Val264 | 264 | 242 | 0.881 | 0.189 | 0.73 | 1.07 | 1.129 | 0.019 | 1.02 | 1.25 |
| 14 | | | | | | | | | | | |
| 15 | | | | | | | | | | | |
| 16 | | | | | | | | | | | |
| 17 | | | | | | | | | | | |
| 18 | | | | | | | | | | | |
| 19 | | | | | | | | | | | |
| 20 | | | | | | | | | | | |
| 21 | | | | | | | | | | | |
| 22 | Val | | | | | | | | | | |
| 23 | | | | | | | | | | | |
| 24 | | | | | | | | | | | |
| 25 | Val | | | | | | | | | | |
| 26 | | | | | | | | | | | |
| 27 | | | | | | | | | | | |
| 28 | | | | | | | | | | | |
| 29 | | | | | | | | | | | |
| 30 | | | | | | | | | | | |
| 31 | Val96 | 118 | | | | | | | | | |
| 32 | Val96* | 214 | | | | | | | | | |
| 33 | | | | | | | | | | | |
| 34 | | | | | | | | | | | |
| 35 | | | | | | | | | | | |
| 36 | | | | | | | | | | | |
| 37 | | | | | | | | | | | |
| 38 | | | | | | | | | | | |
| 39 | Val96 | 258 | | | | | | | | | |
| 40 | | | | | | | | | | | |
| 41 | Val96 | 166 | | | | | | | | | |
| 42 | | | | | | | | | | | |
| 43 | | | | | | | | | | | |
| 44 | | | | | | | | | | | |
| 45 | Val | | | | | | | | | | |
| 46 | Val264 | 221 | 235 | 0.619 | 0.029 | 0.40 | 0.95 | 1.114 | 0.292 | 0.91 | 1.36 |
| 47 | | | | | | | | | | | |
| 48 | | | | | | | | | | | |
| 49 | | | | | | | | | | | |
| 50 | | | | | | | | | | | |
| 51 | | | | | | | | | | | |
| 52 | Val264 | 251 | 246 | 1.206 | 0.393 | 0.79 | 1.85 | 0.996 | 0.974 | 0.80 | 1.24 |
| 53 | | | | | | | | | | | |
| 54 | Val264 | 164 | 242 | 1.373 | 0.070 | 0.98 | 1.93 | 0.912 | 0.258 | 0.78 | 1.07 |
| 55 | | | | | | | | | | | |
| 56 | Val96 | 239 | | | | | | | | | |
| 57 | | | | | | | | | | | |
| 58 | | | | | | | | | | | |
| 59 | Val264 | 191 | 240 | 1.612 | 0.023 | 1.07 | 2.44 | 0.828 | 0.053 | 0.69 | 1.00 |
| 60 | Val | 353 | | | | | | | | | |
| 61 | | | | | | | | | | | |
| 62 | | | | | | | | | | | |
| 63 | | | | | | | | | | | |
| 64 | Val96 | 206 | | | | | | | | | |
| 65 | | | | | | | | | | | |
| 66 | | | | | | | | | | | |
| 67 | | | | | | | | | | | |
| 68 | Val96 | +/−115 | | | | | | | | | |
| 69 | | | | | | | | | | | |
| 70 | | | | | | | | | | | |
| 71 | | | | | | | | | | | |
| 72 | Val264 | 304 | 242 | 1.563 | 0.001 | 1.19 | 2.06 | 0.792 | 0.000 | 0.69 | 0.90 |
| 73 | Val96 | 219 | | | | | | | | | |
| 74 | | | | | | | | | | | |
| 75 | Val96 | 346 | | | | | | | | | |
| 76 | Val264 | 89 | 240 | 1.835 | 0.001 | 1.30 | 2.59 | 0.772 | 0.003 | 0.65 | 0.92 |
| 77 | | | | | | | | | | | |
| 78 | | | | | | | | | | | |
| 79 | Val | | | | | | | | | | |
| 80 | Val264 | 138 | 239 | 1.132 | 0.238 | 0.92 | 1.39 | 0.901 | 0.040 | 0.82 | 1.00 |
| 81 | | | | | | | | | | | |
| Other Markers | | | | | | | | | | | |
| 101 | Val264 | 114 | 235 | 0.612 | 0.004 | 0.44 | 0.86 | 1.263 | 0.006 | 1.07 | 1.49 |
| 102 | Val264 | +/−75 | | | | | | | | | |
| 103 | Val264 | +/−100 | 241 | 1.615 | 0.007 | 1.14 | 2.29 | 0.950 | 0.497 | 0.82 | 1.10 |
| 104 | Val96 | +/−110 | | | | | | | | | |
| 105 | Val264 | +/−80 | 241 | 0.904 | 0.505 | 0.67 | 1.22 | 1.139 | 0.075 | 0.99 | 1.31 |
| 106 | Val96 | +/−105 | | | | | | | | | |
| 107 | Val264 | +/−70 | 241 | 1.307 | 0.041 | 1.01 | 1.69 | 0.997 | 0.962 | 0.88 | 1.13 |
| 108 | Val96 | +/−75 | | | | | | | | | |
| 109 | Val264 | 148 | 246 | 0.846 | 0.328 | 0.61 | 1.18 | 1.176 | 0.066 | 0.99 | 1.40 |
| 110 | Val264 | 133 | | | | | | | | | |
| 111 | Val96 | +/−90 | | | | | | | | | |
| 112 | Val264 | +/−70 | 241 | 1.441 | 0.012 | 1.08 | 1.92 | 0.847 | 0.021 | 0.74 | 0.98 |

[symbol]

indicates data missing or illegible when filed

TABLE 3
Suggested Threshold Values for Predictive Outcome

| | Threshold | Significance |
|---|---|---|
| | Value | Padj |
| Overall Response | | |
| Outcome | | |
| DEME-6 | 9.15 | 0.0096 |
| SIAH2 | 1.16 | 0.0283 |
| CASP2 | 0.94 | 0.0085 |
| THRAP2 | 1.16 | 0.226 |
| FN1 | 140.87 | 0.0701 |
| Progression Free | | |
| Survival Outcome | | |
| DEME-6 | 9.38 | 0.0115 |
| SIAH2 | 0.76 | 0.0206 |
| THRAP2 | 5.02 | 0.385 |
| TNC | 2.08 | 0.254 |

TABLE 4
Regression Analysis of Individual Marker Genes
CUTPOINTS RESPONSE

| | N | OR | P | 95% CI | | HR | P | 95% CI | |
|---|---|---|---|---|---|---|---|---|---|
| Univariate Regression | | | | | | | | | |
| DEME-6 | 240 | 2.97 | <0.001 | 1.65 | 5.38 | 0.60 | <0.001 | 0.45 | 0.79 |
| SIAH2 | 242 | 2.47 | 0.002 | 1.41 | 4.34 | 0.65 | 0.003 | 0.48 | 0.86 |
| CASP2 | 235 | 0.35 | <0.001 | 0.20 | 0.61 | 1.33 | 0.037 | 1.02 | 1.75 |
| Multivariable Regression | | | | | | | | | |
| DEME-6 | 240 | 2.84 | 0.0012 | 1.51 | 5.34 | 0.58 | 0.0002 | 0.43 | 0.77 |
| SIAH2 | 242 | 2.40 | 0.0079 | 1.26 | 4.59 | 0.71 | 0.028 | 0.52 | 0.96 |
| CASP2 | 235 | 0.33 | 0.00044 | 0.18 | 0.61 | 1.39 | 0.022 | 1.05 | 1.85 |

N = number of tumor samples analyzed
OR = Objective Response (OR ≧1 correlates with positive resonse to anti-estrogen therapy)
HR = Hazard Ratio (HR <1 correlates with positive response to anti-estrogen therapy)
P = Significance value; p < 0.05 is desired

TABLE 5
Multivariable Regression Analysis of Marker Gene Combinations

| Marker Genes | N | OR | P | 95% CI | | HR | P | 95% CI | |
|---|---|---|---|---|---|---|---|---|---|
| DEME-6_CASP2 | 231 | | | | | | | | |
| DEME-6 | | 3.08 | 0.00088 | 1.59 | 5.99 | 0.58 | 0.00031 | 0.44 | 0.78 |
| CASP2 | | 0.31 | 0.00029 | 0.16 | 0.58 | 1.42 | 0.015 | 1.07 | 1.89 |
| DEME-6_SIAH2 | 236 | | | | | | | | |
| DEME-6 | | 2.44 | 0.0069 | 1.28 | 4.66 | 0.61 | 0.00088 | 0.46 | 0.82 |
| SIAH2 | | 1.89 | 0.064 | 0.96 | 3.72 | 0.78 | 0.12 | 0.57 | 1.07 |
| CASP2_SIAH2 | 232 | | | | | | | | |
| SIAH2 | | 2.45 | 0.0091 | 1.25 | 4.79 | 0.74 | 0.058 | 0.54 | 1.01 |
| CASP2 | | 0.32 | 0.00045 | 0.17 | 0.61 | 1.35 | 0.036 | 1.02 | 1.80 |

TABLE 6
Multivariable Regression Analysis of Marker Gene Ratios

| Marker Genes | N | OR | P | 95% CI | | HR | P | 95% CI | |
|---|---|---|---|---|---|---|---|---|---|
| DEME-6/CASP2 | 231 | 1.56 | 0.00053 | 1.21 | 2 | 0.85 | 0.0023 | 0.76 | 0.94 |
| DEME-6/SIAH2 | 236 | 0.96 | 0.74 | 0.75 | 1.23 | 1.05 | 0.46 | 0.93 | 1.18 |
| CASP2/SIAH2 | 232 | 0.66 | 0.0005 | 0.53 | 0.84 | 1.19 | 0.00077 | 1.08 | 1.32 |

We claim:

1-18. (canceled)

19. A gene signature predictive of patient response or outcome to anti-estrogen therapy for recurring breast cancer, comprising two or more marker genes identified in Table 1 as differentially expressed in primary tumors of recurring breast cancer patients exhibiting an outcome to anti-estrogen therapy with a significance of p≦0.05.

20. The gene signature of claim 19, wherein said marker genes are selected from the 81-gene signature listed in Table 1.

21. The gene signature of claim 19, wherein said marker genes are selected from the 44-gene signature listed in Table 1.

22. The gene signature of claim 19, wherein said marker genes comprise at least one of FN-1, CASP-2, THRAP-2, SIAH-2, DEME-6, TNC, and COX-6C.

23. The gene signature of claim 19, wherein said marker genes comprise at least one of TNC, SIAH-2, DEME-6, and COX-6C.

24. The gene signature of claim 19, wherein said marker genes comprise at least one of FN-1, CASP-2, THRAP-2, SIAH-2, and DEME-6.

25. The gene signature of claim 19, wherein said marker genes comprise at least one of CASP-2 and DEME-6, and at least one of SIAH-2 and TNC.

26. An assay system for predicting patient response or outcome to anti-estrogen therapy for recurring breast cancer configured and adapted to detect the gene signature of claim 19, comprising:
a) two or more marker genes identified in Table 1 differentially expressed in primary tumors of recurring breast cancer patients exhibiting an outcome to anti-estrogen therapy with a significance of p≦0.05;
b) two or more nucleic acid probes, comprising at least 10 to 50 contiguous nucleic acids of marker genes identified in Table 1 as differentially expressed in primary tumors of recurring breast cancer patients exhibiting an outcome to anti-estrogen therapy with a significance of p≦0.05, or complementary nucleic acid sequences thereof; or
c) two or more binding ligands that specifically detect polypeptides encoded by marker genes identified in Table 1 as differentially expressed in primary tumors of recurring breast cancer patients exhibiting an outcome to anti-estrogen therapy with a significance of p≦0.05.

27. The assay system of claim 26, wherein said marker genes are selected from the 81-gene signature listed in Table 1.

28. The assay system of claim 26, wherein said marker genes are selected from the 44-gene signature listed in Table 1.

29. The assay system of claim 26, wherein said marker genes comprise at least one of FN-1, CASP-2, THRAP-2, SIAH-2, DEME-6, TNC, and COX-6C.

30. The assay system of claim 26, wherein said marker genes comprise at least one of TNC, SIAH-2, DEME-6, and COX-6C.

31. The assay system of claim 26, wherein said marker genes comprise at least one of FN-1, CASP-2, THRAP-2, SIAH-2, and DEME-6.

32. The assay system of claim 26, wherein said marker genes comprise at least one of CASP-2 and DEME-6, and at least one of SIAH-2 and TNC.

33. The assay system of claim 26, wherein said marker genes, nucleic acid probes, or binding ligands are disposed on an assay surface.

34. The assay system of claim 26, wherein said assay surface comprises a chip, array, or fluidity card.

35. The assay system of claim 26, wherein said probes comprise complementary nucleic acid sequences to at least 10 to 50 nucleic acid sequences of said marker genes.

36. The assay system of claim 26, wherein said binding ligands comprise antibodies or binding fragments thereof.

37. A method for predicting outcome of anti-estrogen therapy for recurrent breast cancer, the method comprising:
a) analyzing a patient's primary tumor for expression of two or more marker genes identified in Table 1 as differentially expressed in primary tumors of recurring breast cancer patients exhibiting an outcome to anti-estrogen therapy with a significance of p≦0.05;
b) determining if the expression pattern of said tumor's marker genes correlates with a Cluster 1 or Cluster 2 expression pattern; and
c) correlating a Cluster 1 expression pattern with prediction of Progressive Disease and a Cluster 2 expression pattern with Objective Response to anti-estrogen therapy for recurrent breast cancer.

38. The method of claim 37, wherein said primary tumor is analyzed for expression of the 81-gene signature or the 44-gene signaure listed in Table 1.

39. A method for predicting Progression Free Survival of anti-estrogen therapy for recurrent breast cancer, the method comprising:
a) analyzing a patient's primary tumor for expression of two or more marker genes identified in Table 1 as differentially expressed in primary tumors of recurring breast cancer patients exhibiting an outcome to anti-estrogen therapy with a significance of p≦0.05;
b) determining if the expression pattern of said tumor's marker genes correlates with a Cluster 1 or Cluster 2 expression pattern; and
c) correlating a Cluster 1 expression pattern with a negative prediction of Progression Free Survival for recurrent breast cancer and a Cluster 2 expression pattern with a positive Progression Free Survival for recurrent breast cancer.

40. The method of claim 39, wherein said primary tumor is analyzed for expression of the the 81-gene signature or the 44-gene signaure listed in Table 1.
